(19) United States
(12) Patent Application Publication
(10) Pub. No.: US 2026/0315708 A1
(43) Pub. Date: Oct. 15, 2026

(54) A DELIVERY SYSTEM WITH DOSE DIVIDER

(71) Applicant: Sanofi Pasteur, Inc., Swiftwater, PA (US)

(72) Inventor: Stephan Riedel, Frankfurt (DE)

(21) Appl. No.: 19/482,124

(22) PCT Filed: May 10, 2024
(86) PCT No.: PCT/US2024/028786

§ 371 Date: Nov. 6, 2025

(30) Foreign Application Priority Data
May 11, 2023 (EP) 23172970.8

(51) Int. Cl. A61M 11/00; A61M 15/08

(52) U.S. Cl. CPC A61M 11/007; A61M 15/08

(57) ABSTRACT

A delivery system comprises a syringe having a chamber for containing medicament and an outlet through which medicament can be expelled. A plunger rod is moveable relative to the syringe and configured to cause medicament to be expelled from the syringe when medicament is within the syringe and the plunger rod is moved. A disc, when the delivery system is in a first configuration, permits the plunger rod to move over a first range of motion relative to the syringe to expel a first dose of medicament, and blocks the plunger rod from moving over a subsequent range of motion relative to the syringe to expel further medicament. The disc or the plunger rod is rotatable to put the delivery system into a second configuration, in which the plunger rod is free to move over the subsequent range of motion relative to the syringe to expel further medicament

### TECHNICAL FIELD

The present disclosure relates to a medicament delivery system configured to facilitate delivery of a medicament in separate doses.

### BACKGROUND

Respiratory syncytial virus (RSV) is an important cause of severe acute lower respiratory illness (LRI) in infants and children and a common cause of severe pneumonia requiring hospital admission in children worldwide. According to global estimates, RSV caused approximately 33 million cases of LRI and approximately 118 000 deaths in children <5 years of age in 2015. Greater than 80% of all RSV-associated LRIs (RSV-LRIs) and more than 50% of the RSV-associated deaths in low-and middle-income countries were estimated to occur in infants ≥6 months old.

Medicament delivery devices may typically comprise a syringe of medicament having an outlet through which the medicament can be expelled when a plunger rod is displaced through the syringe.

Some medicaments are intended to be delivered in two or more doses from a single syringe. In such cases, a user may be required to manipulate the plunger rod over a first range of motion to dispense a first dose, and then manipulate the plunger rod over one or more subsequent range(s) of motion to dispense subsequent doses. Some patients may have difficulty in manipulating the plunger rod to accurately dispense the intended separate doses with the intended quantities of medicament in each dose. Alternatively, patients may accidentally dispense unintended quantities of medicament, or dispense all of the medicament at once, through inexperience with the device or misunderstanding of device usage instructions.

### SUMMARY

In a first aspect of the disclosure, there is provided a delivery system comprising:
a syringe comprising a chamber for containing medicament and an outlet through which medicament can be expelled from the chamber;
a plunger rod moveable relative to the syringe and configured to cause medicament to be expelled from the syringe when medicament is within the syringe and the plunger rod is moved; and
a disc which, when the device is in a first configuration, permits the plunger rod to move over a first range of motion relative to the syringe to expel a first dose of medicament, and blocks the plunger rod from moving over a subsequent range of motion relative to the syringe to expel further medicament;
wherein either the disc or the plunger rod is rotatable to put the device into a second configuration, in which the plunger rod is free to be moved over the subsequent range of motion relative to the syringe to expel further medicament.

Therefore the plunger rod is easily manipulated to accurately dispense intended separate doses with the intended quantities of medicament in each dose. Further, patients or other device operators are prevented from accidentally dispensing unintended quantities of medicament, or dispensing all of the medicament at once.

In some embodiments, the disc is configured to be rotated relative to the syringe to put the delivery system in the second configuration.

In some embodiments, the plunger rod comprises a first region and a second region.

In some embodiments, the first and second regions are integrally formed.

In some embodiments, the first and second regions together form an elongate member of the plunger rod.

The disc may have an opening through which the plunger rod is displaced to dispense medicament, wherein the plunger rod has a first region formed to permit the first region to pass through the opening in the disc when the plunger rod or the disc is in a first rotational orientation about a longitudinal axis of the device to dispense the first dose of medicament, and wherein the plunger rod has a second region proximal to the first region, the second region being formed to prevent the second region from passing through the opening when the plunger rod or disc are in the first rotational orientation.

The second region may be formed to permit the second region to pass through the opening in the disc when the plunger rod or disc is in a second rotational orientation about the longitudinal axis of the device to dispense a second dose of medicament.

The medicament delivery device may further be configured to deliver more than two doses of medicament, wherein the plunger rod comprises further regions in addition to the first and second region, each further region being formed to pass through the opening in the disc only when the plunger rod or the disc is in a predetermined rotational orientation about the longitudinal axis associated with said further region.

The disc may be releasably attached to a finger flange of the syringe.

Therefore the disc may be reused should the syringe be disposed of.

The disc may be configured to rotate about a longitudinal axis of the device between a first position, in which the disc permits the plunger rod to move over the first range of motion and blocks the plunger rod from moving over the subsequent range of motion, and a second position, in which the plunger rod is free to be moved over the subsequent range of motion relative to the syringe to expel further medicament.

The device may further comprise a guide element, the guide element having an opening through which the plunger rod is movable to dispense medicament, the opening being configured to prevent rotation of the plunger rod about the longitudinal axis of the device.

The disc may comprise an opening that overlaps the opening of the guide element, and wherein, when the disc is in the first position, the plunger rod is permitted to move over the first range of motion through the opening of the disc and the opening of the guide element, but blocked from moving over the subsequent range of motion; and wherein, when the disc is in the second position, the plunger rod is permitted to move through the subsequent range of motion through the opening of the disc and the opening of the guide element to expel further medicament.

The plunger rod may comprise a first region at a distal end of the plunger rod and a second region at a proximal end of the plunger rod, and wherein the cross-sectional profiles of the first and second regions are the same and comprise rotational symmetry of order n, where n is an integer; and wherein the cross-sectional profile of the first and second regions are offset by an angle α about a longitudinal axis of the plunger rod. Optionally, α≠360°/n.

Therefore, either the disc or the plunger may be rotated to move from the first configuration into the second configuration.

The plunger rod may comprise a first region at a distal end of the plunger rod and a second region at a proximal end of the plunger rod, and wherein the first region comprises a first cross-sectional profile and the second region comprises a second cross-sectional profile, different to the first cross-sectional profile.

The opening in the guide element may be shaped as a superimposition of the offset cross-sectional profile of the first and second regions of the plunger rod.

The opening in the disc may be shaped to match the cross-sectional profile of the first and second regions of the plunger rod.

The opening in the guide element may be shaped as a superimposition of the first and second cross-sectional profiles of the plunger rod.

The opening in the disc may be shaped as a superimposition of the first and second-cross-sectional profiles of the plunger rod.

The plunger rod may comprise a third region disposed in between the first and second regions, and wherein the third region comprises a cross-sectional profile shaped so that, when the third region is located within the opening of the disc, the disc is free to rotate.

Therefore the delivery device is configured to constrain the plunger rod throughout its range of motion, the device being moved from the first configuration into the second configuration by rotation of the disc.

The plunger rod may be configured to rotate about a longitudinal axis of the device between a first position in which the disc permits the plunger rod to move over the first range of motion, and blocks the plunger rod from moving over the subsequent range of motion, and a second position, in which the plunger rod is free to be moved over the subsequent range of motion relative to the syringe to expel further medicament.

The plunger rod may comprise an elongate element slidable linearly over its range of motion.

The plunger rod may comprise a plunger configured to compress a reservoir of medicament.

The plunger rod may comprise a piston rod slidable linearly into the syringe.

The syringe may comprise a barrel and an outlet at a distal end of the barrel.

The syringe may comprise a piston in sealing contact with a barrel wall and slidable within the barrel by movement of the plunger rod to expel medicament from the outlet.

The plunger rod may be configured such that substantially half of the medicament in the chamber is expelled as the plunger rod moves over the first range of motion, and substantially half of the medicament in the chamber is expelled as the plunger rod moves over the second range of motion.

The medicament delivery device may further comprise a plug at the outlet of the device to facilitate insertion of the plug and outlet into an orifice of a patient into which the medicament is to be delivered.

The plug and/or outlet may comprise a Luer-Lock to sealingly connect the plug to the outlet.

Therefore the device is configured to the nasal administration of medicament, such as the administration of a vaccine.

The plug may be configured to atomise a liquid medicament as the medicament is expelled through the outlet and through a medicament passage in the plug.

Medicament may be received within the chamber of the syringe.

The syringe may be provided with a medicament.

The medicament may be a vaccine adapted for nasal administration, the vaccine may be an RSV vaccine.

The device may be an intranasal atomization delivery device.

The device may be configured to deliver about ½ of a dose to each nostril.

The device may be configured to deliver about 0.2 mL, wherein about 0.1 mL is delivered to each nostril.

The intranasal atomization delivery device may deliver an average droplet size Dᵥ₅₀ of about 10-120 μm.

The intranasal atomization delivery device may deliver an average droplet size Dᵥ₅₀ delivered to each nostril may be about 10-120 μm, about 30-110 μm, about 50-110 μm, about 70-110 μm, or about 80-110 μm.

The intranasal atomization delivery device may be configured so that an average shot volume to each nostril is between about 85 μL to about 120 μL, about 90 μL to about 115 μL, or about 95 μL to about 115 μL.

In a second aspect of the disclosure, there is provided apparatus comprising a finger flange attachment and a plunger rod for use with a syringe, the finger flange attachment comprising a disc which, when attached to a finger flange of a syringe is configurable to permit the plunger rod to move over a first range of motion relative to the syringe to expel a first dose of medicament, and block the plunger rod from moving over a subsequent range of motion relative to the syringe to expel further medicament.

Therefore a syringe may be simply adapted to provide the advantages of the present disclosure.

The disc may be rotatable into a second configuration, in which the plunger rod is permitted to be moved over the subsequent range of motion relative to the syringe to expel further medicament.

In some embodiments, the disc is configured to be rotated relative to the syringe to put the delivery system in a second configuration.

In a third aspect of the disclosure, there is provided a method of operating a delivery system as described above, the method comprising:
inserting the outlet into a first nostril of a patient;
moving the plunger rod over a first range of motion relative to the syringe to expel a first dose of medicament;
putting the device in a second configuration;
inserting the outlet into a second nostril of a patient; and
moving the plunger rod over the subsequent range of motion relative to the syringe to expel further dose of medicament.

In some embodiments, putting the device in the second configuration comprises rotating a disc relative to a syringe.

The method may comprise administering an RSV vaccine.

The delivery system may comprise an intranasal atomisation delivery device.

The RSV vaccine may be delivered intranasally with about ½ dose delivered to each nostril.

The intranasal dose of the RSV vaccine may be delivered in about 0.2 mL, wherein about 0.1 mL is delivered to each nostril.

The intranasal atomization delivery device may deliver an average droplet size Dᵥ₅₀ of about 10-120 μm.

The intranasal atomization delivery device may deliver an average droplet size Dᵥ₅₀ delivered to each nostril may be about 10-120 μm, about 30-110 μm, about 50-110 μm, about 70-110 μm, or about 80-110 μm.

The intranasal atomization delivery device may be configured so that an average shot volume to each nostril is between about 85 μL to about 120 μL, about 90 μL to about 115 μL, or about 95 μL to about 115 μL.

According to the present disclosure, there is also provided a delivery system comprising: a syringe comprising a chamber for containing medicament and an outlet through which medicament can be expelled from the chamber; a plunger rod moveable relative to the syringe and configured to cause medicament to be expelled from the syringe when medicament is within the syringe and the plunger rod is moved; and a disc which, when the delivery system is in a first configuration, permits the plunger rod to move over a first range of motion relative to the syringe to expel a first dose of medicament, and blocks the plunger rod from moving over a subsequent range of motion relative to the syringe to expel further medicament; wherein the disc is rotatable relative to the syringe to put the delivery system into a second configuration, in which the plunger rod is free to be moved over the subsequent range of motion relative to the syringe to expel further medicament.

In some embodiments, the plunger rod comprises a first region and a second region and wherein the disc has an opening through which the plunger rod is displaced to dispense medicament, wherein the first region of the plunger rod is formed to permit the first region to pass through the opening in the disc when the disc is in a first rotational orientation about a longitudinal axis of the device to dispense the first dose of medicament, and wherein the second region of the plunger rod is proximal to the first region, the second region being formed to prevent the second region from passing through the opening when the disc is in the first rotational orientation, and wherein the second region is formed to permit the second region to pass through the opening in the disc when the disc is in a second rotational orientation about the longitudinal axis of the device to dispense a second dose of medicament.

These and other aspects of the disclosure will be apparent from and elucidated with reference to the embodiments described hereinafter.

### BRIEF DESCRIPTION OF THE FIGURES

Embodiments will now be described, by way of example only, with reference to the accompanying drawings, in which:

FIG. 1 is a perspective view of a medicament delivery device in accordance with an example of the disclosure, shown in an initial position;

FIG. 2 is a side view of the device of FIG. 1;

FIG. 3 is a side view of the device of FIG. 1 after the plunger rod has been moved over a first range of motion;

FIG. 4 is a side view of the device of FIG. 1 after the plunger rod has been moved over a second range of motion;

FIG. 5 is a perspective view of a plunger rod for use in examples of the disclosure;

FIG. 6 is detail view of a disc and finger grip for use in examples of the disclosure;

FIG. 7 is a detail view of a disc and finger grip for use in examples of the disclosure;

FIG. 8 is detail view of a disc and finger grip for use in examples of the disclosure;

FIG. 9 is a perspective view of a plunger rod for use in examples of the disclosure;

FIG. 10 is detail view of a disc and finger grip for use in examples of the disclosure;

FIG. 11 is a detail view of a disc and finger grip for use in examples of the disclosure;

FIG. 12 is detail view of a disc and finger grip for use in examples of the disclosure;

FIG. 13 is a perspective view of a plunger rod for use in examples of the disclosure;

FIG. 14 is detail view of a disc and finger grip for use in examples of the disclosure;

FIG. 15 is a detail view of a disc and finger grip for use in examples of the disclosure;

FIG. 16 is detail view of a disc and finger grip for use in examples of the disclosure;

FIG. 17a shows finger flange attachment for use in examples of the disclosure;

FIG. 17b shows a syringe and plunger rod for use with the finger flange attachment of FIG. 17a; and

FIG. 17c shows a medicament delivery in accordance with an example of the disclosure, the medicament delivery device having been assembled from the finger flange attachment of FIG.

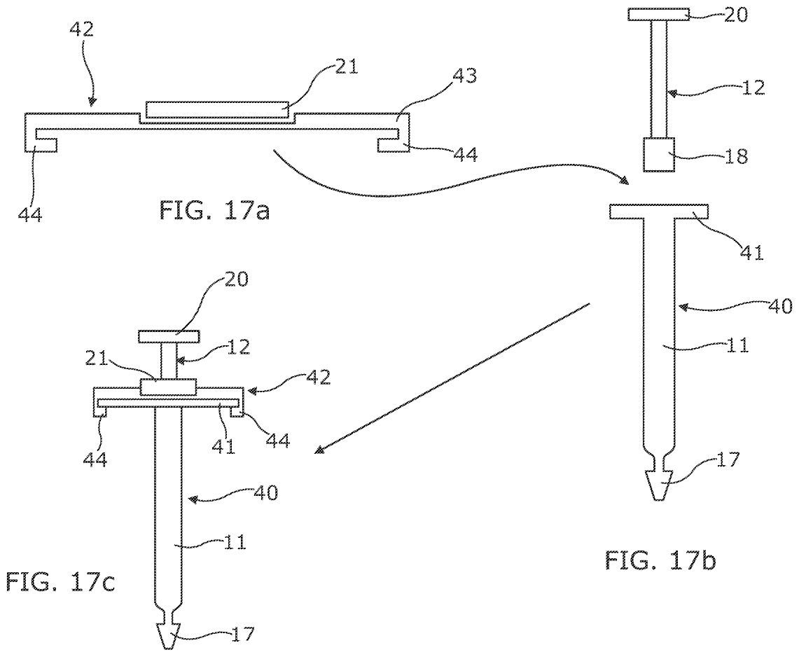

17a and the syringe and plunger rod of FIG. 17b.

### DETAILED DESCRIPTION

Described herein are systems, devices, components, and methods that may be associated with delivering a respiratory syncytial virus (RSV) vaccine using an intranasal atomization delivery device.

As used herein, “RSV ΔNS2/Δ1313/I1314L” refers to an RSV ΔNS2/Δ1313/I1314L (NIH) or an RSV ΔNS2/Δ1313/I1314L (Sanofi). Each of the ΔNS2/Δ1313/I1314L (NIH) and the RSV ΔNS2/Δ1313/I1314L (Sanofi) comprise a live-attenuated RSV with (i) a 523 nucleotide (nt) deletion of the NS2 gene (ΔNS2), (ii) an amino acid deletion in the L protein, and (iii) a genetically stabilizing mutation in the L gene. The live-attenuated RSV of the RSV ΔNS2/Δ1313/Δ1314L (Sanofi) also includes a nucleotide modification at position 14456 that represents a change from a thymine (T) to an adenine (A) in a non-coding region.

As used herein, “RSV ΔNS2/Δ1313/I1314L vaccine” refers to an “RSV ΔNS2/Δ1313/I1314L (NIH) vaccine” or an “RSV ΔNS2/Δ1313/I1314L (Sanofi) vaccine.” An RSV ΔNS2/Δ1313/I1314L (NIH) vaccine comprises an effective amount of RSV ΔNS2/Δ1313/I1314L (NIH). An RSV ΔNS2/Δ1313/I1314L (Sanofi) vaccine comprises an effective amount of RSV ΔNS2/Δ1313/I1314L (Sanofi).

### Exemplary Methods and Uses

In some embodiments, provided are methods of delivering a dose of an RSV vaccine using an intranasal atomization delivery device.

In some embodiments, the methods of administering a dose of an RSV vaccine that comprises a live attenuated RSV use an intranasal atomization delivery device. In some embodiments, provided are methods of administering a dose of an RSV vaccine using an intranasal atomization delivery device, the RSV vaccine comprising an effective amount of a live-attenuated RSV with (i) a 523 nucleotide (nt) deletion of the NS2 gene (ΔNS2), (ii) an amino acid deletion in the L protein, and (iii) a genetically stabilizing mutation in the L gene (RSVΔ NS2/Δ1313/I1314L) (NIH) vaccine) or RSVΔ NS2/Δ1313/I1314L (Sanofi) vaccine that further comprises a nucleotide modification in a non-coding region that represents a change from a thymine (T) to an adenine (A). In some embodiments, a codon that encodes a serine at position 1313 of the L protein is deleted resulting in the deletion of the amino acid in the L protein (Δ1313). In some embodiments, an amino acid residue substitution of leucine for isoleucine at position 1314 results in a genetically stabilizing mutation in the L gene (I1314L).

In some embodiments, provided are methods of administering a dose of an RSV vaccine using an intranasal atomization delivery device. In some embodiments, the pediatric subject may be 6 to 18 months of age.

In some embodiments, the RSV vaccine is delivered intranasally using the intranasal atomization delivery device to deliver about ½ dose to each nostril. In some embodiments, the RSV vaccine is delivered intranasally so that the whole dose is delivered to one nostril. In some embodiments, the RSV vaccine is delivered intranasally with ½ dose delivered to one nostril and the other ½ dose is delivered to the same nostril after the first ½ dose is absorbed. In some embodiments, the RSV vaccine is delivered intranasally using the intranasal atomization delivery device to deliver a dose in unequal amounts to one or both nostrils.

In some embodiments, the RSV vaccine is delivered intranasally using the intranasal atomization delivery device in a liquid formulation. In some embodiments, the RSV vaccine dose is delivered intranasally using the intranasal atomization delivery device in about 0.2 mL. In some embodiments, the 0.2 mL dose is delivered intranasally wherein about 0.1 mL is delivered to each nostril. In some embodiments, the RSV vaccine dose is delivered intranasally using the intranasal atomization delivery device in about 0.01 mL, 0.02 mL, 0.05 mL, 0.1 mL, 0.2 mL, 0.3 mL, 0.4 mL, 0.5 mL, 0.6 mL, 0.7 mL, 0.8 mL, 0.9 mL, 1.0 mL, 1.1 mL, 1.2 mL, 1.3 mL, 1.4 mL or 1.5 mL. As described above, the dose may be divided evenly between two nostrils, divided unevenly between two nostrils, or delivered all to one nostril in one or more deliveries.

In some embodiments, provided are methods of administering a first dose of an RSV vaccine using an intranasal atomization delivery device and administering a second dose of the RSV vaccine using an intranasal atomization delivery device.

In some embodiments, provided are methods of administering a first dose of an RSV vaccine using an intranasal atomization delivery device wherein the intranasal atomization delivery device comprises a spray nozzle to atomize the RSV vaccine and direct a spray plume toward a top of a nasal passageway into a nasal cavity. In some embodiments, provided are methods of administering a dose of an RSV vaccine using an intranasal atomization delivery device wherein the intranasal atomization delivery device comprises a spray nozzle to atomize the RSV vaccine and direct a spray plume toward a top of a nasal passageway into a nasal cavity. In some embodiments, the intranasal atomization delivery device comprises a barrel operably connected to the spray nozzle and a plunger movable within the barrel to advance the RSV vaccine through the spray nozzle. In some embodiments, the intranasal atomization delivery device further includes a dose divider for splitting the dose of the RSV vaccine into two or more deliveries. In some embodiments, the dose divider may divide the dose of the RSV vaccine in about half of a volume to be delivered to a subject. In some embodiments the dose divider is used to deliver ½ dose to each nostril of the subject.

In some embodiments, the intranasal atomization delivery device delivers an average droplet size Dᵥ₅₀ of 10-120 μm. In some embodiments, the intranasal atomization delivery device delivers an average droplet size Dᵥ₅₀ of 10-120 μm, about 30-110 μm, about 50-110 μm, about 70-110 μm, or about 80-110 μm. In some embodiments, the intranasal atomization delivery device delivers an average shot weight between about 95 mg to about 135 mg, between about 100 mg to about 130 mg, or between about 100 mg to about 130 mg or between about 105 mg to about 130. In some embodiments, the intranasal atomization delivery device delivers an average shot volume of about 85 μL to about 120 μL, about 90 μL to about 115 μL, or about 95 μL to about 115 μL.

In some embodiments, use of an intranasal atomization delivery device described herein for administering a dose of an RSV vaccine to a subject is provided wherein the RSV vaccine comprises an effective amount of a live-attenuated RSV. In some embodiments, the use includes a live-attenuated RSV with (i) a 523 nucleotide (nt) deletion of the NS2 gene (ΔNS2), (ii) an amino acid deletion in the L protein, and (iii) a genetically stabilizing mutation in the L gene (RSVΔ NS2/Δ1313/I1314L (NIH) vaccine) or RSVΔ NS2/Δ1313/I1314L (Sanofi) vaccine that further comprises a nucleotide modification in a non-coding region that represents a change from a thymine (T) to an adenine (A).

### Immunogenicity

Approximately seventy percent (70%) of vaccine recipients attained a 4-fold response in neutralizing antibody titer following the second vaccine dose in both low and high dose groups, compared to 61 and 47% following one vaccine dose in low and high dose RSV naïe participants. This increase in the percentage of participants attaining a 4-fold response following a second administration supports the use of a second vaccine administration in this population. The 70% attaining this fold rise post second vaccine administration aligns with the expected clinical efficacy goal of 70% for the candidate. After one or two vaccinations, 75% attained a 4-fold rise in serum neutralizing antibody titers. The percentage of RSV experienced participants (36% and 22% in the low and high dose groups respectively) also attaining a 4-fold response in neutralizing antibody titers post vaccination 1 suggests potential benefit for this sub-group as well. It must be noted that this comes from a modest sample of participants (n=20 of 97 vaccine recipients) at this point of interim analysis.

Taken together, these data strongly support the use of RSV ΔNS2/Δ1313/I1314L (Sanofi) vaccine at an operative range study for the candidate including doses at 5.6 and 6.2 log PFU/dose.

### Vaccine Virus Shedding and Infectivity

Following each vaccine administration, the shedding of vaccine virus was considered in addition to the fold rise in neutralizing antibody titers or serum IgG as vaccine infectivity. The high level of vaccine infectivity (over 80% and 70% in RSV naïve following the first and second vaccine administration respectively) is supportive of a promising vaccine candidate associated with good vaccine take. When infectivity was considered after either vaccine administration, over 90% of participants had evidence of infection. In the small cohort of RSV experienced participants, relatively high infectivity (80% and 33.3% in low and high dose recipients following one vaccine administration and 60 and 50% following a second vaccine administration) was found, implicating promise for this group. In addition, the marked drop in the percentage of vaccine virus shedders in RSV naïve participants after the second vaccine administration (roughly 20%) compared to the first vaccine administration (over 70%) is as previously documented with other efficacious live attenuated mucosal viral vaccines where subsequent ‘challenge’ in the form of a second vaccine dose is characterized by a marked reduction in vaccine virus shedding. Of note, the shedding data available for this cohort was from data at a single timepoint following each vaccination (seven days post vaccination). While this coincides with the point of peak viral shedding documented in other RSV live attenuated vaccine (LAV) trials, it is likely that some shedders may have been missed. This limitation in the available shedding data makes the results obtained particularly encouraging.

### Overall Conclusions

Interim analysis results showed a promising safety, immunogenicity and infectivity profile of the RSV ΔNS2/Δ1313/I1314L (Sanofi) candidate.

No safety concerns were identified after 1- and 2-dose administrations of either dose level of the investigational RSV ΔNS2/Δ1313/I1314L (Sanofi) vaccine or by baseline serostatus.

The vaccine virus shedding, and immunogenicity conclusions based on the IgA serostatus at baseline show marked vaccine take demonstrated at both dose levels, and 70% of vaccine RSV-naïve recipients attained a 4-fold response in serum neutralizing antibody responses post the second vaccine administration for both dose levels in RSV-naïve participants.

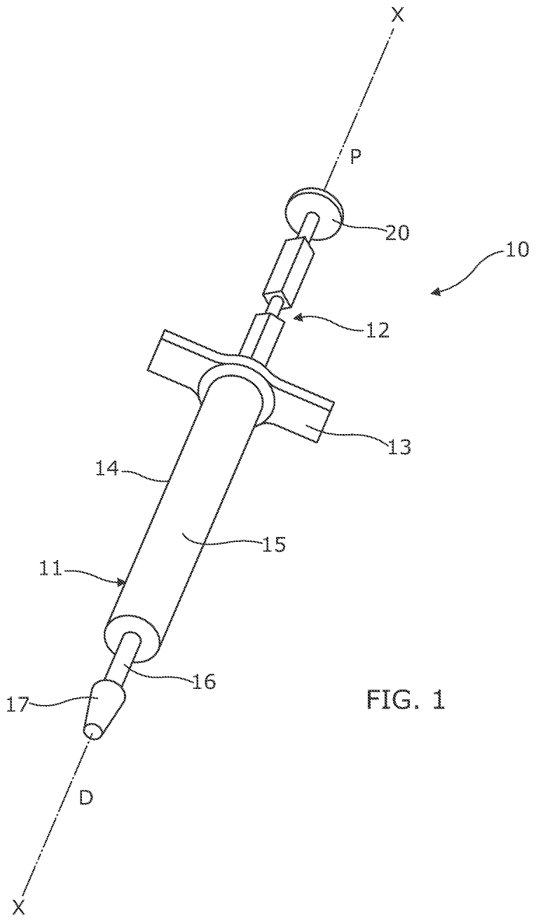

FIGS. 1 to 4 show a medicament delivery device 10 (hereafter “device 10”) according to a first embodiment. The device 10 has a proximal end P and a distal end D, and is generally elongate with a central axis X-X. The device 10 comprises a medicament container 11 (hereafter “container 11”), a plunger rod 12, and finger grip 13 secured to a proximal end of the container 11. In embodiments described herein, the container 11 is a syringe 11 and the finger grip 13 a finger flange 13 of the syringe 11. The terms ‘syringe 11’ and ‘container 11’ may be used herein interchangeably. The container 11 is elongate and comprises an outer side wall 14 defining a medicament chamber 15. The container 11 is open at a proximal end P and includes an outlet 16 at the distal end D opposite to the proximal end of the container 11. The term “distal” refers to a location that is relatively closer to the outlet 16 and from which medicament is delivered in use, and the term “proximal” refers to a location that is relatively further away from the outlet 16. A distal region of the plunger rod 12, received within the container 11, is in sealing contact with the inside wall of the container 11. The proximal end of the plunger rod 12 includes an end surface 20 which, in use, a user would press a thumb against to dispense medicament (described in more detail below).

The outlet 16 comprises a narrowing of the container into a nozzle. A plug 17 is provided on the outlet 16. The plug 17 and/or outlet 16 may comprise a Luer-Lock to sealingly connect the plug 17 to the outlet 16. In some embodiments, the plug 17 may be formed to facilitate insertion of the outlet into an orifice into which medicament is to be delivered. Optionally, the plug 17 may be configured with a medicament passage configured to atomise a liquid medicament expelled through the outlet 16 and through the plug 17.

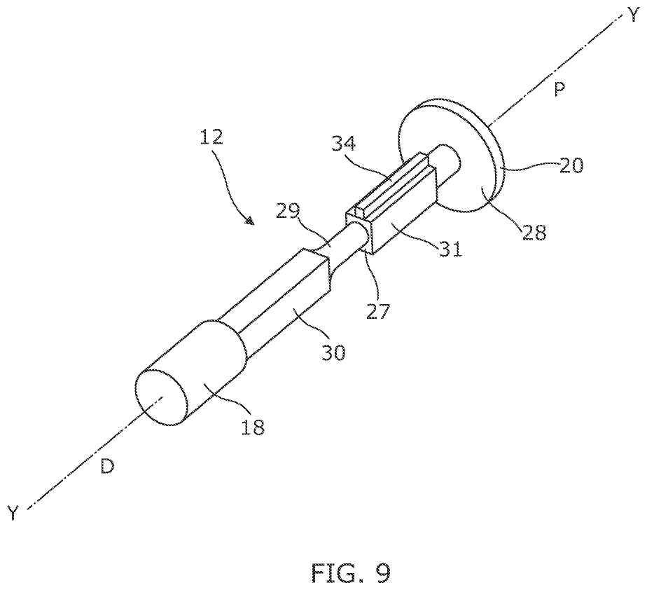

In the exemplary embodiment shown in FIGS. 1 to 4, the device 10 is generally in the form of a syringe where the container 11 comprises a syringe barrel and the plunger rod 12 is received within the syringe barrel. In some embodiments, a piston 18 may be provided within the medicament chamber 15 in sealing contact with the inside wall of the container 11. In such an arrangement, the piston 18 may be attached to the distal end of the plunger rod 12 (see FIG. 9), and the plunger rod 12 may therefore not need to make sealing contact with the inside wall of the container 11.

In use, medicament 19 would be provided within the medicament chamber 15, and would remain in the chamber 15 due to the seal of the plunger rod 12 and/or piston 18 at the proximal end of the chamber 15, and the narrow outlet 16 at the distal end of the chamber 15, which may be provided with a cap, valve, temporary rupturable seal, or other closure. Alternatively, the narrow outlet 16 alone may prevent medicament 19 escaping from the distal end of the chamber 15. The medicament 19 may be expelled in one or more doses from the chamber 15 by linearly moving the plunger rod 12 in a distal direction into the chamber 15 to reduce the volume of the chamber and force the medicament 19 out of the outlet 16.

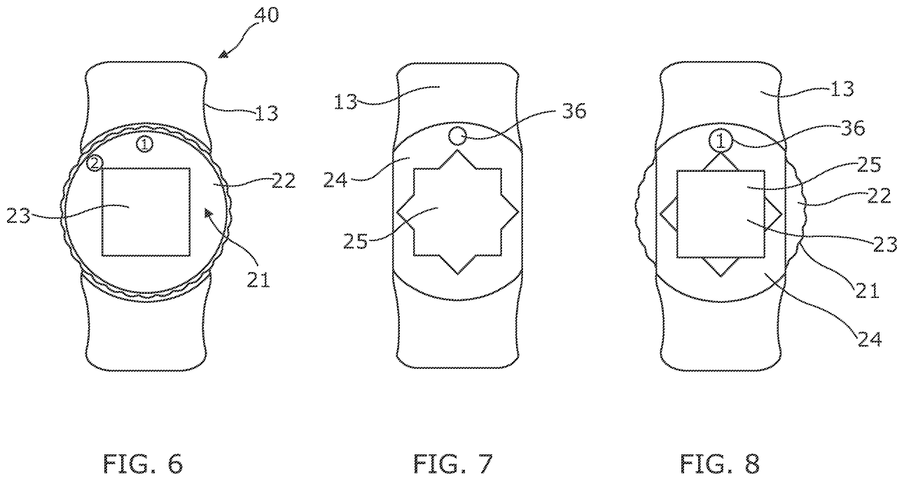

The device 10 includes a disc 21 which functions as a “dose divider” in use of the device. The disc, or dose divider 21 (“disc” hereafter) is shown in more detail in FIG. 6 and comprises a body 22 with an opening 23 extending through the body 22.

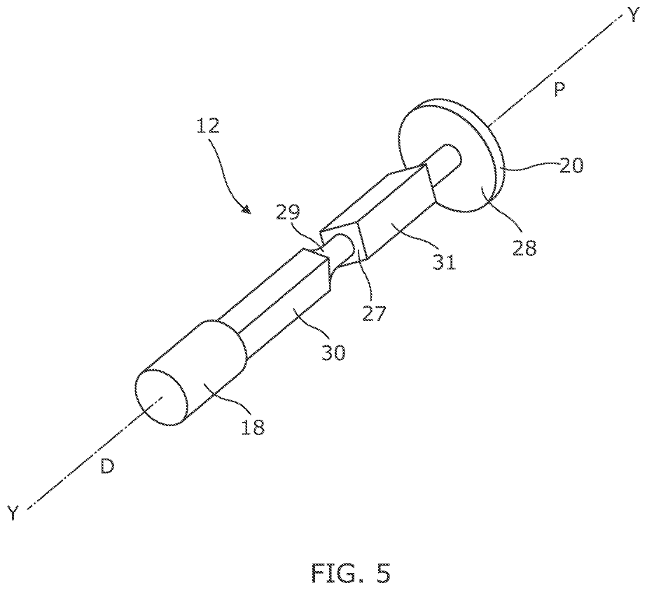

The plunger rod 12 is shown in more detail in FIG. 5 and comprises an elongate rod with an optional piston or piston 18, so the plunger rod 12 serves as a piston rod or plunger rod for moving the piston 18 within the container 11. In various embodiments, the piston 18 may be formed integrally with the plunger rod 12, or may be mechanically or otherwise (such as by adhesive bonding) attached to the plunger rod 12, or the plunger rod 12 may abut against the piston 18. The plunger rod 12 comprises a first region 30 at a distal end of the plunger rod 12 and a second region 31 at a proximal end of the plunger rod. The first and second regions of the plunger rod 12 comprise a square cross-sectional profile. The cross-sectional profiles of the first and second regions 30, 31 are offset from each other by 45 degrees about a longitudinal axis Y-Y of the plunger rod 12.

The plunger rod 12 is arranged to slide through the opening 23 in the disc 21. The opening 23 is aligned with the axis X-X of the bore of the medicament chamber 15, so the plunger rod 12 extends through the opening 23 and into the container 11. It will be appreciated that the device 10 and plunger rod 12 axes X-X, Y-Y are collinear when the device is assembled, that is to say, the plunger rod 12 is aligned with the longitudinal axis X-X of the device. The opening 23 in the disc 21 is shaped to match the cross-sectional profile of the first and second regions of the plunger rod 12, but is slightly larger in each dimension to allow the plunger rod 12 to freely slide though the opening 23.

In an initial position, the first region 30 of the plunger rod is disposed in the opening 23 of the disc 21. Because the cross-sectional profiles of the first and second sections 30, 31 of the plunger rod 12 are offset about the longitudinal axis Y-Y of the plunger rod 12, either the plunger rod 12 or the disc 21 is required to rotate about the longitudinal axis Y-Y of the plunger rod before the second region 31 of the plunger rod can pass through the opening 23 of the disc 21. Where the disc 21 is fixed, then the plunger rod 12 can be rotated. For example, the disc 21 can be fixed to the finger grip 13. In such an example (not shown), the disc 21 may be integrally formed with the finger grip 13, the opening 23 being provided in the finger grip 13.

Alternatively, and as illustrated by FIGS. 6 to 8, the disc 21 is rotatable and a guide element 24 is provided to prevent rotation of the plunger rod 12. The guide element 24 comprises an opening 25 that overlaps the opening in the disc 21, as shown in FIG. 8. Therefore, the plunger rod extends through both openings 23, 25 of the disc 21 and guide element. 23. The opening 25 of the guide element 24 is shaped as a superimposition of the offset cross-sectional profile of the first and second regions 30, 31 of the plunger rod 12. This means that, irrespective of whether the first or second region 30, 31 of the plunger rod is disposed within the guide element 24, the plunger rod 12 is prevented from rotating about its axis Y-Y due to interference between the plunger rod 12 and the opening 25 of the guide element 24. The plunger rod 12 remains constrained by the guide element 24 throughout the plunger rod's 12 range of motion. That is to say, irrespective of the position of the plunger rod 12, either the first or second region 30, 31 of the plunger rod is disposed within the opening 25 of the guide element 24. As illustrated by FIGS. 6 to 8, the guide element 24 may be integral with the finger grip 13, the opening 25 of the guide element 24 being an opening 25 in the finger grip 13. In such examples, the disc 21 comprises a disc shaped body 22 that sits in a correspondingly shaped recess of the finger grip 13 to allow rotation of the disc 21.

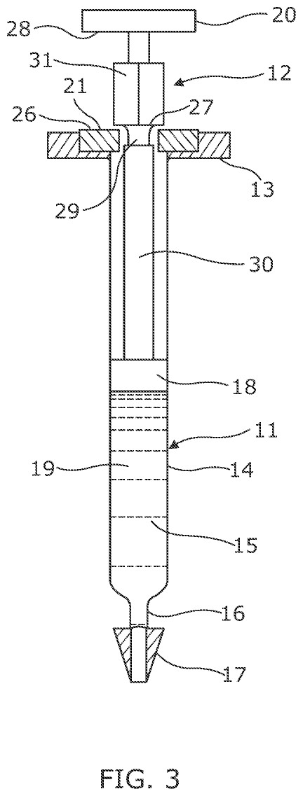

The plunger rod 12 further comprises a third region 29 in between the first and second regions 30, 31. In an intermediate position of the plunger rod 12—as shown in FIG. 3—the third region 29 of the plunger rod 12 is disposed within the opening 23 of the disc 21. The third region is shaped so as not to interfere with the opening 23 in the disc 21 to allow rotation of either the plunger rod 12 or the disc 21, as required.

Operation of the device 10 of the above-described embodiment will now be described. The operation of the device 10 will be described in the context of use as an intranasal medicament delivery device 10, that is, a device for delivery of medicament into the nostril(s) of a patient.

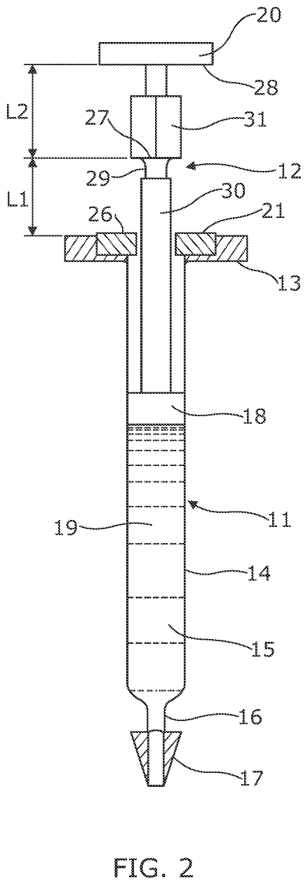

However, the concept is not intended to be limited to intranasal devices and may equally be applicable to various other forms of medicament delivery devices in which the medicament is intended to be delivered in two or more separate doses. In the context of an intranasal device, the volume of medicament in the device 10, for example a vaccine, if often required to be delivered into each nostril, often with the volume of medicament divided equally into a dose for each nostril. In use of such a device, the device 10 starts in the initial position shown in FIG. 2. Here, the plunger rod 12 is in its start position and none of the medicament 19 has been expelled from within the chamber 15. Also in this initial position, the first region 30 of the plunger rod 12 is disposed within the opening 23 of the disc 21. In this position, the disc 21 and the plunger rod 12 are said to be in a ‘first position’ and the device 10 in a ‘first configuration’.

The user (which may be a patient themselves if self-administered, or a healthcare professional or other user if the medicament is being administered to the patient by a third party) inserts the plug 17 into the first nostril. The user then presses the end surface 20 with their thumb, with their index and middle fingers on the finger grip 13, to move the plunger rod 12 axially in a distal direction D. This pushes the piston 18 distally within the chamber 15 and expels the medicament 19 out of the outlet 16 and through the medicament passage of the plug 17 to atomise the medicament for inhalation by the patient. The user continues to move the plunger rod 12 distally over this first range of motion until a distal face 27 of the second region 31 abuts a proximal face 26 of the disc 21 and the plunger rod 12 therefore cannot move any further in the distal direction. At this point, the plunger rod 12 is in the intermediate position and a first dose of the medicament 19 has been delivered to the patient. That is, the plunger rod 12 is in the intermediate position having been moved over a first range of motion but there remains some medicament 19 within the chamber 15 for subsequent dose(s) to be delivered. Also at this point, first region 30 of the plunger rod 12 has moved into the container 11 and now the third region 29 of the plunger rod 12 is positioned within the opening 23 of the disc 21. Since the third region 29 is shaped so as not to interfere with the opening 23 in the disc 21, either the plunger rod 12 or the disc 21 may now be rotated. Where a guide element 24 is provided, the plunger rod 12 is prevented from being rotated and the disc 21 is instead rotatable.

The user then removes the device 10 from the first nostril and rotates either the disc 21 or the plunger rod 12 into a second position, putting the device 10 into a second configuration. In the second configuration, the opening 23 in the disc 21 is aligned with the second region 31 of the plunger rod 12 so as not to obstruct further movement of the plunger rod 12. Put another way, the distal face 27 of the second region 31 and the proximal face 26 of the disc 21 are moved out of abutting relation. The user then inserts the plug 17 into the other nostril. Since the disc 21 or plunger rod 12 has been rotated, the plunger rod 12 is no longer prevented from moving further axially in the distal direction. The user then presses the end surface 20 again to move the plunger rod 12 axially in a distal direction D which pushes the piston 18 distally within the chamber 15 and expels the remaining medicament 19 out of the outlet 16 and through the medicament passage of the plug 17 to atomise the medicament for inhalation by the patient.

The user continues to move the plunger rod 12 distally over this second range of motion until either a distal face 28 of the end surface 20 abuts the proximal face 26 of the disc 21, or the piston 18 abuts the distal end of the chamber 15 and therefore cannot move any further in the distal direction. At this point, the plunger rod 12 is in an end position and the remaining medicament 19 has been expelled as a second and final dose delivered to the patient.

The device 10 described above provides a simple and effective way to ensure a user is able to divide the medicament delivery process into two separate doses, and the disc 21 provides an effective way to ensure the medicament 19 is divided as intended and the user cannot accidentally deliver too much/all of the medicament in the first dose. Furthermore, since the disc 21 is not removable from the finger grip 13, the disc 21 cannot be tampered with. This further helps towards ensuring the medicament is delivered in the intended dose volumes in each dose.

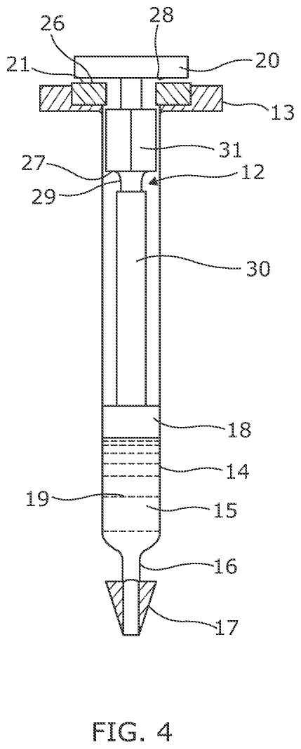

As can be seen from FIG. 2, the distance between the proximal face 26 of the disc 21 and the distal face 27 of the second region 31 of the plunger rod 12 has a length dimension in the axial direction of L1. In the exemplary embodiment shown, this length L1 also corresponds to the length L2 in the axial direction of the second region 31 of the plunger rod 12. It will be appreciated that the volume of medicament 19 delivered in the first dose is determined by the distance the plunger rod 12 is able to move from the initial position (shown in FIG. 2) to the intermediate position (shown in FIG. 3), which is limited by when the second region 31 of the plunger rod 12 abuts the disc 21. That is, this volume of medicament of the first dose is determined by the distance L1 over which the plunger rod 12 moves in the first range of motion. It will also be appreciated that the volume of medicament 19 delivered in the second dose is determined by the remaining distance the plunger rod 12 is able to move from the intermediate position (shown in FIG. 3) to the end position (shown in FIG. 4), which is limited by when the end surface 20 abuts the disc 21, which may also coincide with when the piston 18 abuts the distal end of the chamber 15. That is, this volume of medicament of the second dose is determined by the distance L2 over which the plunger rod 12 moves in the second range of motion.

In view of the above, the ratio of volumes of first and second doses of medicament to be delivered can be controlled by controlling the first and second ranges of motion of the plunger rod 12, which may be controlled by selecting dimensions L1 and L2 of the device 10 as necessary. In embodiments where the first and second doses are to be equal, it will be appreciated that dimensions L1 and L2 can be made the same.

While the operation of the device 10 is described above with reference to a plunger rod 12 substantially as shown in FIG. 5, it will be appreciated that other plunger rods 12 may be provided having first and second regions 30, 31 with a different shape cross-sectional profile.

The cross-sectional profile of the first and second regions 30, 31 may be any suitable shape that, when offset about the longitudinal axis Y-Y of the plunger rod 12, causes the distal face 27 of the second region 31 of the plunger rod 12 to abut the proximal face 26 of the disc 21 when the plunger rod 12 is moved from the initial position to the intermediate position. Therefore, any shape comprising rotational symmetry of order n, where n is an integer may be suitable. The cross-sectional profile of the first and second regions 30, 31 must be offset by an angle α about a longitudinal axis of the plunger rod 12, wherein α≠360°/n.

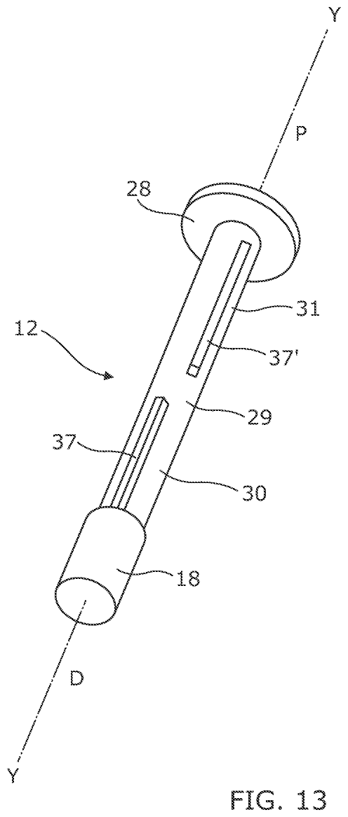

An alternative plunger rod 12 is shown in FIG. 13, with like features retaining the same reference numbers. The plunger rod of FIG. 13 differs from the plunger rod 12 described above only in that the plunger rod 12 comprises a cylindrical cross-section of constant diameter along all of the first, second, and third regions 30, 31, 29. The cross-sections of the first and second regions 30, 31 each further comprise a portion 37, 37′ that extends outside of the cylindrical cross-section. Said portions are herein referred to as first and second keys 37, 37′. The first key 37 is offset from the second key 37′ about the longitudinal axis Y-Y of the plunger rod by an angle that does not equal 360 degrees. Therefore, including the keys 37, 37′, the first and second regions 30, 31 comprise the same cross-sectional area, but offset along the longitudinal axis Y-Y.

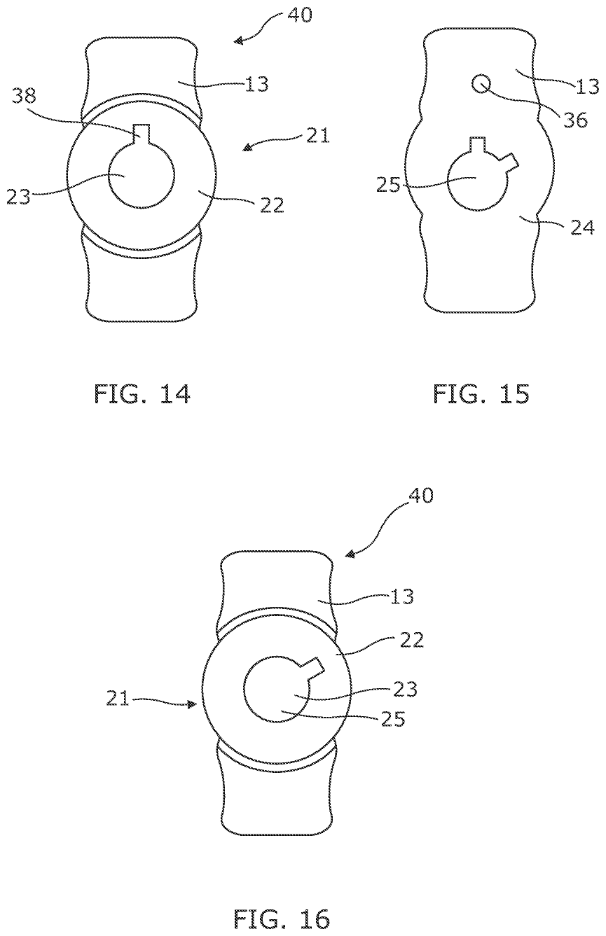

A disc 21 and guide element 24 for use with the plunger rod 12 of FIG. 13 is shown in FIGS. 14 to 16. The opening 23 in the disc 21 comprises a keyway 38 that allow the keys 37, 37′ of the first and second regions 30, 31 to pass through only when aligned therewith. Therefore, just as described above in connection with FIGS. 5 to 8, the opening 23 in the disc 21 is shaped to match the cross-sectional profile of the first and second regions 30, 31 of the plunger rod 12.

Also as above, the opening 25 in the guide element 24 is shaped as a superimposition of the offset cross-sectional profile of the first and second regions 30, 31 of the plunger rod 12. It will therefore be appreciated that the guide element 24 and disc 21 function substantially as described in connection with FIGS. 5 to 8. That is to say, when the device 10 is in the first configuration, the disc 21 permits the plunger rod 12 to move over a first range of motion relative to expel a first dose of medicament, and blocks the plunger rod 12 from moving over a subsequent range of motion; the disc 21 being rotatable about the third region 29 of the plunger rod 12 to put the device 10 into a second configuration in which the plunger rod 12 is free to be moved over the subsequent range of motion to expel further medicament. As above, at all times the guide element 24 prevents rotation of the plunger rod 12.

It will be appreciated that the constant diameter cross-section of the plunger rod 12 of FIG. 13 affords better stress distribution than other plunger rod designs and therefore better longevity.

This is because the stress tends to concentrate in areas where the cross-section changes, which can reduce the number of cycles of operation that may be achieved before fatigue failure. It will also be appreciated that by ‘constant diameter cross-section’, the plunger rod 12 is not necessarily limited to a circular cross-section, but can be any constant cross-sectional area shape that can move through openings 23, 25 of the guide element 24 and disc 21 unobstructed. Evidently, the constant cross-sectional area does not include the keys 37, 37′, which provide the necessary interference with the disc 21. It is also important that the constant cross-sectional area shape does not obstruct rotation of the disc 21 when the third region 29 is disposed therein.

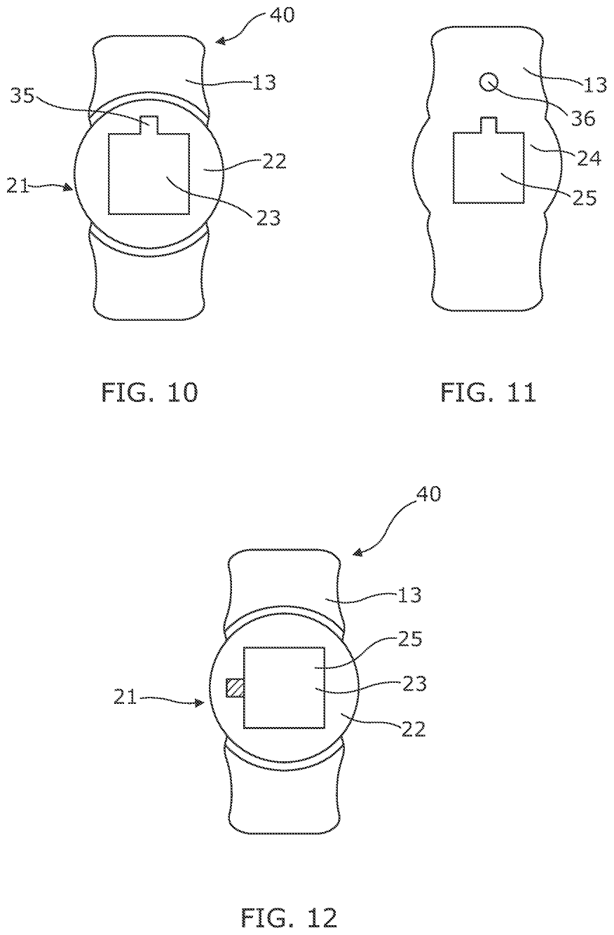

Another alternative plunger rod 12 is shown in FIG. 9, with like features retaining the same reference numbers. The plunger rod of FIG. 9 differs from the plunger rods 12 described above only in that the cross-sectional profiles of the first and second regions 30, 31 of the plunger rod are not the same. That is, the first region 30 comprises a first cross-sectional profile that is different to the cross-sectional profile of the second region 31. In FIG. 12, the cross-sectional profile of the first region 30 is square, while the cross-sectional profile of the second region 31 comprises a portion 34 that extends outside of the first cross-sectional profile, as projected along the axis Y-Y. A disc 21 and guide element 24 for use with the plunger rod 12 of FIG. 9 is shown in FIGS. 10 to 12. The opening 23 in the disc 21 is shaped to match the cross-sectional profile of the second region 31 of the plunger rod and, therefore, comprises a region 35 that corresponds with the portion 34 of the second cross-sectional profile. As before, the opening 23 is slightly larger in each dimension to allow the plunger rod 12 to freely slide though the opening 23. The guide element 24 is again optional, but where included, the opening 25 of the guide element is shaped to match the cross-sectional profile of the second region 31 of the plunger rod 12.

For the second region 31 of the plunger rod 12 to pass through the opening 23 in the disc 21, the portion 34 of the second region 31 must be aligned with region 35 of the opening 23 in the disc 21. In the initial position, region 35 of the opening 23 in the disc 21 is misaligned with the portion 34 of the second region 31 of the plunger rod 12. Therefore, as before, either the plunger rod 12 or the disc 21 is required to rotate about the longitudinal axis Y-Y of the plunger rod 12 before the second region 31 of the plunger rod can pass through the opening 23 of the disc 21. Where the disc 21 is fixed, then the plunger rod 12 can be rotated. Where the guide element 24 is provided, the disc 21 can be rotated so that the respective openings 23, 25 in the disc 21 and the guide element 24 are aligned, as shown in FIG. 10. Thus, in every other respect, a device 10 comprising a plunger rod 12 as shown in FIG. 9 is operated in precisely the same way as the device 10 comprising a plunger rod 12 as shown in FIG. 5 and a detailed description thereof will not be repeated.

The device 10 may further comprise a counter 36 as illustrated in FIG. 8 by way of example. The counter 36 comprises a window 36 in a distal face of the finger grip 13 through which a portion of the distal face of the disc 21 is visible. Said visible portion of the distal face of the disc 21 may be provided with a number that is visible through the window 36. For example, with the disc 21 in the first position, the number ‘2’ may be visible through the window, rotation of the disc 21 from the first position into the second position causing the number ‘1’ to instead be displayed. Therefore, in the first position, the counter 36 may indicate the number of doses remaining in the medicament chamber 15.

The device 10 may further comprise a clicker mechanism (not shown) to positively locate the disc 21 in the first or second position. The clicker provides haptic feedback to allow a user to determine when the disc 21 is in either the first or second position. The clicker may be configured in any suitable way. For example, the distal face of the disc 21 and the proximal face of the guide element 24 may comprise a series of protrusions and indents, respectively.

Therefore, the protrusions may locate in the indents when the disc 21 is in either of the first or second positions.

A medicament delivery device as described above may be formed by providing a finger flange attachment 42 and a syringe 11 having an integrated finger flange 41 and attaching the finger flange attachment 42 to the finger flange 41 of the syringe 11. The finger flange attachment 42 and is illustrated in FIG. 17a. The finger flange attachment 42 comprises the guide element 24 and, optionally, the disc 21. The disc 21 may take the form of any of the above described embodiments. In order to form the medicament delivery device, it is further necessary to provide a plunger rod 12 configured to operate in accordance with the description above. That is to say, a plunger rod 12 having first and second regions 30, 31 that cooperate with the guide element 24 and (optionally) the disc 21 so that, when the device 10 is in the first configuration, the plunger rod 12 is permitted to move over a first range of motion to expel a first dose of medicament, but blocked from moving over a subsequent range of motion, the disc 21 or the plunger rod 12 being rotatable to put the device 10 into a second configuration in which the plunger rod 12 is free to be moved over the subsequent range of motion to expel further medicament.

The finger flange attachment 42 comprises means to allow the finger flange attachment 42 to be clipped or otherwise attached to the finger flange 41 of the syringe 11. In FIG. 17a, the finger flange attachment 42 comprises a body 43 in which the guide element 24 is formed. The body 43 further comprises C shaped ends 44 which are configured to fit over free ends of the finger flange 41. The C shaped ends 44 may be configured for a tight fit with the free ends of the finger flange 41 to securely locate the finger flange attachment 42 to the syringe 11.

A method of assembling the medicament delivery device is explained with reference to FIGS. 17a through 17c. The method comprises: providing the finger flange attachment 42 of FIG. 17a; providing a syringe 11 and plunger rod 12 (as exemplified by the illustration of FIG. 17b); attaching the finger flange attachment 42 to the finger flange 41 of the syringe 11; and inserting the plunger rod 12 into a medicament container 11 of the syringe 11.

It shall be appreciated that the present disclosure may be configured to deliver more than two doses of medicament. For example, a straightforward adaptation of the plunger shown in FIG. 5 would allow for the controlled delivery of three or more doses by the inclusion of additional regions of the plunger rod 12. Each region would comprise the same square section profile, but offset from the last by 45 degrees. The additional regions would be separated by intermediate regions corresponding in form and function to the third region 29 shown in FIG. 5. Therefore, when the intermediate regions are located in the opening 23 of the disc 21, the disc 21 or plunger rod 12 can be rotated by 45 degrees into alignment with profile of the following adjacent region to allow said following adjacent region to pass through the disc 21.

It will be appreciated that the present disclosure may be adapted for a form of medicament delivery other than nasal administration. For example, the embodiments described herein may take the form of a needle based injection device by replacement of the plug 17 with a hypodermic needle. The hypodermic needle may comprise a Luer-Lock to attach to the outlet 16 of the syringe in the usual way. By replacement of the plug 17 with the hypodermic needle, the embodiments described herein may be adapted for subcutaneous, intramuscular or intravenous injection, depending on the medicament to be delivered and the gauge of needle used.

### REFERENCE NUMERAL KEY

10 medicament delivery device
11 medicament container
12 plunger rod
13 finger grip
14 outer side wall of container
15 medicament chamber
16 outlet
17 plug
18 piston
19 medicament
20 end surface
21 disc
22 body of the disc
23 opening through the disc
24 guide element
25 opening through guide element
26 proximal face of the disc
27 distal face of the second region of the plunger rod
28 distal face of the end surface
29 third region of the plunger rod
30 first region of plunger rod
31 second region of plunger rod
34 portion of the second cross-sectional profile
35 region of the opening in the disc corresponding to portion 34
36 counter/window in distal face of finger grip
37 key of the first region
37′ key of the second region
38 keyway
40 medicament delivery device comprising a finger flange attachment separate to a syringe
41 finger flange
42 finger flange attachment 43 finger flange body
44 C shaped ends of the finger flange body

According to the following clauses, there is disclosed:

1. A delivery system (10) comprising:
a syringe (11) comprising a chamber (15) for containing medicament and an outlet (16) through which medicament (19) can be expelled from the chamber;
a plunger rod (12) moveable relative to the syringe and configured to cause medicament to be expelled from the syringe when medicament is within the syringe and the plunger rod is moved; and
a disc (21) which, when the device is in a first configuration, permits the plunger rod to move over a first range of motion relative to the syringe to expel a first dose of medicament, and blocks the plunger rod from moving over a subsequent range of motion relative to the syringe to expel further medicament;
wherein either the disc (21) or the plunger rod (12) is rotatable to put the device into a second configuration, in which the plunger rod is free to be moved over the subsequent range of motion relative to the syringe to expel further medicament.

2. A system according to clause 1, wherein the disc (21) has an opening (23) through which the plunger rod (12) is displaced to dispense medicament, wherein the plunger rod (12) has a first region (30) formed to permit the first region (30) to pass through the opening (23) in the disc (21) when the plunger rod (12) or the disc (21) is in a first rotational orientation about a longitudinal axis (X-X) of the device (10) to dispense the first dose of medicament, and wherein the plunger rod (12) has a second region (31) proximal to the first region (30), the second region (31) being formed to prevent the second region (31) from passing through the opening (23) when the plunger rod (12) or disc (21) are in the first rotational orientation; and, optionally, wherein the second region (31) is formed to permit the second region (31) to pass through the opening (23) in the disc (21) when the plunger rod (12) or disc (21) is in a second rotational orientation about the longitudinal axis (X-X) of the device (10) to dispense a second dose of medicament; and, optionally, wherein the system is further configured to deliver more than two doses of medicament, wherein the plunger rod comprises further regions in addition to the first and second region, each further region being formed to pass through the opening in the disc only when the plunger rod or the disc is in a predetermined rotational orientation about the longitudinal axis associated with said further region.

3. A system (10) according to clause 1, wherein the disc (21) is configured to rotate about a longitudinal axis of the device (10) between a first position, in which the disc (21) permits the plunger rod to move over the first range of motion and blocks the plunger rod from moving over the subsequent range of motion, and a second position, in which the plunger rod is free to be moved over the subsequent range of motion relative to the syringe to expel further medicament; and, optionally, wherein the system further comprises a guide element (24), the guide element having an opening (25) through which the plunger rod (12) is movable to dispense medicament, the opening being configured to prevent rotation of the plunger rod about the longitudinal axis of the device; and, optionally, wherein the disc (21) comprises an opening (23) that overlaps the opening (25) of the guide element (24), and wherein, when the disc is in the first position, the plunger rod (12) is permitted to move over the first range of motion through the opening of the disc and the opening of the guide element, but blocked from moving over the subsequent range of motion; and wherein, when the disc is in the second position, the plunger rod is permitted to move through the subsequent range of motion through the opening of the disc and the opening of the guide element to expel further medicament; and, optionally, wherein the plunger rod (12) comprises a first region (30) at a distal end of the plunger rod and a second region (31) at a proximal end of the plunger rod, and wherein the cross-sectional profiles of the first and second regions are the same and comprise rotational symmetry of order n, where n is an integer; and wherein the cross-sectional profile of the first and second regions are offset by an angle α about a longitudinal axis of the plunger rod; and, optionally, wherein α≠360°/n; and, optionally, wherein the plunger rod (12) comprises a first region (30) at a distal end of the plunger rod and a second region (31) at a proximal end of the plunger rod, and wherein the first region comprises a first cross-sectional profile and the second region comprises a second cross-sectional profile, different to the first cross-sectional profile; and, optionally, wherein the opening (25) in the guide element (24) is shaped as a superimposition of the offset cross-sectional profile of the first and second regions of the plunger rod (12); and, optionally, wherein the opening (23) in the disc (21) is shaped to match the cross-sectional profile of the first and second regions of the plunger rod; and, optionally, wherein the opening (25) in the guide element (24) is shaped as a superimposition of the first and second cross-sectional profiles of the plunger rod (12); and, optionally, wherein the opening (23) in the disc (21) is shaped as a superimposition of the first and second cross-sectional profiles of the plunger rod (12); and, optionally, wherein the plunger rod (12) comprises a third region (29) disposed in between the first and second regions (30, 31), and wherein the third region comprises a cross-sectional profile shaped so that, when the third region is located within the opening of the disc (21), the disc is free to rotate.

4. A system (10) according to clause 1, wherein the plunger rod (12) is configured to rotate about a longitudinal axis of the device (10) between a first position in which the disc (21) permits the plunger rod to move over the first range of motion, and blocks the plunger rod from moving over the subsequent range of motion, and a second position, in which the plunger rod is free to be moved over the subsequent range of motion relative to the syringe to expel further medicament.

5. A system (10) according to any preceding clause, wherein the plunger rod (12) comprises an elongate element slidable linearly over its range of motion; and, optionally, wherein the plunger rod (12) comprises a plunger configured to compress a reservoir (32) of medicament; and, optionally, wherein the plunger rod (12) comprises a piston rod slidable linearly into the syringe (11).

6. A system (10) according to clause 2 wherein the syringe comprises a barrel and an outlet (16) at a distal end of the barrel.

7. A system (10) according to clause 16 wherein the syringe comprises a piston (18) in sealing contact with a barrel wall and slidable within the barrel by movement of the plunger rod (12) to expel medicament (19) from the outlet (16).

8. A system (10) according to any preceding clause, wherein the plunger rod (12) is configured such that substantially half of the medicament (19) in the chamber (15) is expelled as the plunger rod moves over the first range of motion, and substantially half of the medicament in the chamber is expelled as the plunger rod moves over the second range of motion.

9. A system (10) according to any preceding clause, further comprising a plug (17) at the outlet (16) of the device to facilitate insertion of the plug and outlet into an orifice of a patient into which the medicament is to be delivered.

10. A system (10) according to clause 9, wherein the plug (17) is configured to atomise a liquid medicament (19) as the medicament is expelled through the outlet (16) and through a medicament passage in the plug.

11. A system (10) according to any preceding clause, wherein medicament (19) is received within the chamber (15) of the syringe (11).

12. A system (10) according to any preceding clause, wherein the syringe is provided with a medicament and, optionally, wherein the medicament is a vaccine adapted for nasal administration and, optionally, wherein the vaccine is an RSV vaccine.

13. Apparatus comprising a finger flange attachment (42) and a plunger rod (12) for use with a syringe, the finger flange attachment comprising a disc (21) which, when attached to a finger flange of a syringe is configurable to permit the plunger rod to move over a first range of motion relative to the syringe to expel a first dose of medicament, and block the plunger rod from moving over a subsequent range of motion relative to the syringe to expel further medicament.

14. Apparatus according to clause 13, wherein the disc (21) is rotatable into a second configuration, in which the plunger rod is permitted to be moved over the subsequent range of motion relative to the syringe to expel further medicament.

15. A method of operating a delivery system (10), the delivery system (10) comprising:
a syringe (11) comprising a chamber (15) for containing medicament and an outlet (16) through which medicament (19) can be expelled from the chamber;
a plunger rod (12) moveable relative to the syringe and configured to cause medicament to be expelled from the syringe when medicament is within the syringe and the plunger rod is moved; and
a disc (21) which, when the device is in a first configuration, permits the plunger rod to move over a first range of motion relative to the syringe to expel a first dose of medicament, and blocks the plunger rod from moving over a subsequent range of motion relative to the syringe to expel further medicament;
wherein either the disc (21) or the plunger rod (12) is rotatable to put the device into a second configuration, in which the plunger rod is free to be moved over the subsequent range of motion relative to the syringe to expel a further dose of medicament;
the method comprising:
inserting the outlet (16) into a first nostril of a patient;
moving the plunger rod (12) over a first range of motion relative to the syringe (11) to expel the first dose of medicament;
putting the device in the second configuration;
inserting the outlet into a second nostril of a patient; and
moving the plunger rod over the subsequent range of motion relative to the syringe to expel the further dose of medicament.

1. A delivery system (10) comprising:
a syringe (11) comprising a chamber (15) for containing medicament and an outlet (16) through which medicament (19) can be expelled from the chamber;
a plunger rod (12) moveable relative to the syringe and configured to cause medicament to be expelled from the syringe when medicament is within the syringe and the plunger rod is moved; and
a disc (21) which, when the delivery system is in a first configuration, permits the plunger rod to move over a first range of motion relative to the syringe to expel a first dose of medicament, and blocks the plunger rod from moving over a subsequent range of motion relative to the syringe to expel further medicament;
wherein the disc (21) or the plunger rod (12) is rotatable to put the delivery system into a second configuration, in which the plunger rod is free to be moved over the subsequent range of motion relative to the syringe to expel further medicament.

2. A system according to claim 1, wherein the plunger rod (12) comprises a first region (30) and a second region (31) and, optionally, wherein the first and second regions are integrally formed.

3. A system (10) according to claim 2, wherein the first region (30) of the plunger rod (12) is at a distal end of the plunger rod and the second region (31) of the plunger rod (12) is at a proximal end of the plunger rod, and wherein cross-sectional profiles of the first and second regions are the same and comprise rotational symmetry of an order n, where n is an integer; and wherein the cross-sectional profiles of the first and second regions are offset by an angle α about a longitudinal axis of the plunger rod; and, optionally, wherein the angle α does not equal 360°/n.

4. A system (10) according to claim 2, wherein the first region (30) of the plunger rod (12) is at a distal end of the plunger rod and the second region (31) of the plunger rod (12) is at a proximal end of the plunger rod, and wherein the first region comprises a first cross-sectional profile and the second region comprises a second cross-sectional profile different than the first cross-sectional profile.

5. A system (10) according to any one of claims 2 to 4, wherein the disc (21) comprises an opening (23) through which the plunger rod (12) is displaced to dispense medicament.

6. A system (10) according to claim 5, wherein the opening (23) in the disc (21) is shaped to match the cross-sectional profile of the first and second regions of the plunger rod, or wherein the opening (23) in the disc (21) is shaped as a superimposition of the first and second cross-sectional profiles of the plunger rod (12).

7. A system according to claim 5 or claim 6, wherein the first region (30) of the plunger rod (12) is formed to permit the first region (30) to pass through the opening (23) in the disc (21) when the plunger rod (12) or the disc (21) is in a first rotational orientation about a longitudinal axis (X-X) of the delivery system (10) to dispense the first dose of medicament, and wherein the second region (31) of the plunger rod (12) is proximal to the first region (30), the second region (31) being formed to prevent the second region (31) from passing through the opening (23) when the plunger rod (12) or disc (21) are in the first rotational orientation.

8. A system according to claim 7, wherein the second region (31) is formed to permit the second region (31) to pass through the opening (23) in the disc (21) when the plunger rod (12) or disc (21) is in a second rotational orientation about the longitudinal axis (X-X) of the delivery system (10) to dispense a second dose of medicament.

9. A system according to any one of claims 5 to 8, wherein the delivery system is configured to deliver more than two doses of medicament, wherein the plunger rod comprises further regions in addition to the first and second region, each further region being formed to pass through the opening in the disc only when the plunger rod or the disc is in a predetermined rotational orientation about a longitudinal axis associated with the further region.

10. A system (10) according to any one of the preceding claims, wherein the disc is configured to rotate relative to the syringe to put the delivery system into the second configuration.

11. A system (10) according to any one of the preceding claims, wherein the disc (21) is configured to rotate about a longitudinal axis of the delivery system (10) between a first position, in which the disc (21) permits the plunger rod to move over the first range of motion and blocks the plunger rod from moving over the subsequent range of motion, and a second position, in which the plunger rod is free to be moved over the subsequent range of motion relative to the syringe to expel further medicament.

12. A system (10) according to any one of the preceding claims, comprising a guide element (24) having an opening (25) through which the plunger rod (12) is movable to dispense medicament, the opening being configured to prevent rotation of the plunger rod about a longitudinal axis of the delivery system.

13. A system (10) according to claim 12, when dependent on claim 5, wherein opening (23) of the disc (21) overlaps the opening (25) of the guide element (24), and wherein when the disc is in the first position, the plunger rod (12) is permitted to move over the first range of motion through the opening of the disc and the opening of the guide element and is blocked from moving over the subsequent range of motion; and
wherein when the disc is in the second position, the plunger rod is permitted to move through the subsequent range of motion through the opening of the disc and the opening of the guide element to expel further medicament.

14. A system (10) according to claim 12 or claim 13, when dependent on claim 2, wherein the opening (25) in the guide element (24) is shaped as a superimposition of offset cross-sectional profiles of the first and second regions of the plunger rod (12) or wherein the opening (25) in the guide element (24) is shaped as a superimposition of the first and second cross-sectional profiles of the plunger rod (12).

15. A system (10) according to any one of claims 1 to 10, wherein the plunger rod (12) is configured to rotate about a longitudinal axis of the delivery system (10) between a first position in which the disc (21) permits the plunger rod to move over the first range of motion, and blocks the plunger rod from moving over the subsequent range of motion, and a second position, in which the plunger rod is free to be moved over the subsequent range of motion relative to the syringe to expel further medicament.

16. A system (10) according to any one of the preceding claims, wherein the plunger rod (12) comprises a third region (29) disposed in between the first and second regions (30, 31), and wherein the third region comprises a cross-sectional profile shaped so that, when the third region is located within the opening of the disc (21), the disc is free to rotate.

17. A system (10) according to any one of the preceding claims, wherein the plunger rod (12) comprises an elongate element slidable linearly over its range of motion; and, optionally, wherein the plunger rod (12) comprises a plunger configured to compress a reservoir (32) of medicament; and, optionally, wherein the plunger rod (12) comprises a piston rod slidable linearly into the syringe (11).

18. A system (10) according to any one of the preceding claims, wherein the syringe comprises a barrel and the outlet (16) at a distal end of the barrel and, optionally wherein the syringe comprises a piston (18) in sealing contact with a barrel wall and slidable within the barrel by movement of the plunger rod (12) to expel medicament (19) from the outlet (16).

19. A system (10) according to any one of the preceding claims, wherein the plunger rod (12) is configured such that substantially half of the medicament (19) in the chamber (15) is expelled as the plunger rod moves over the first range of motion, and substantially half of the medicament in the chamber is expelled as the plunger rod moves over the second range of motion.

20. A system (10) according to any one of the preceding claims, further comprising a plug (17) at the outlet (16) of the delivery system to facilitate insertion of the plug and outlet into an orifice of a patient into which the medicament is to be delivered and, preferably, wherein the plug (17) is configured to atomise a liquid medicament (19) as the medicament is expelled through the outlet (16) and through a medicament passage in the plug.

21. A system (10) according to any one of the preceding claims, wherein medicament (19) is received within the chamber (15) of the syringe (11).

22. A system (10) according to any one of the preceding claims, wherein the syringe is provided with a medicament and, optionally, wherein the medicament is a vaccine adapted for nasal administration and, optionally, wherein the vaccine is an RSV vaccine.

23. An apparatus comprising:
a finger flange attachment (42), and
a plunger rod (12) for use with a syringe,
wherein the finger flange attachment comprises a disc (21) which, when attached to a finger flange of a syringe is configurable to permit the plunger rod to move over a first range of motion relative to the syringe to expel a first dose of medicament, and block the plunger rod from moving over a subsequent range of motion relative to the syringe to expel further medicament.

24. An apparatus according to claim 23, wherein the disc (21) is rotatable into a second configuration, in which the plunger rod is permitted to be moved over the subsequent range of motion relative to the syringe to expel further medicament.

25. A method of operating a delivery system (10), the delivery system (10) comprising:
a syringe (11) comprising a chamber (15) for containing medicament and an outlet (16) through which medicament (19) can be expelled from the chamber;
a plunger rod (12) moveable relative to the syringe and configured to cause medicament to be expelled from the syringe when medicament is within the syringe and the plunger rod is moved; and
a disc (21) which, when the delivery system is in a first configuration, permits the plunger rod to move over a first range of motion relative to the syringe to expel a first dose of medicament, and blocks the plunger rod from moving over a subsequent range of motion relative to the syringe to expel further medicament;
wherein the disc (21) or the plunger rod (12) is rotatable to put the delivery system into a second configuration, in which the plunger rod is free to be moved over the subsequent range of motion relative to the syringe to expel a further dose of medicament;
the method comprising:
inserting the outlet (16) into a first nostril of a patient;
moving the plunger rod (12) over a first range of motion relative to the syringe (11) to expel the first dose of medicament;
putting the delivery system in the second configuration;
inserting the outlet into a second nostril of a patient; and
moving the plunger rod over the subsequent range of motion relative to the syringe to expel the further dose of medicament.

26. A method of operating a delivery system, the method comprising:
inserting an outlet (16) of a syringe (11) into a first nostril of a patient;
moving a plunger rod (12) over a first range of motion relative to the syringe to expel a first dose of medicament;
rotating a disc (21) relative to the plunger rod to put the delivery system into a second configuration;
inserting the outlet into a second nostril of the patient; and
moving the plunger rod over a subsequent range of motion relative to the syringe to expel a further dose of medicament.
